(19) United States
(12) Patent Application Publication
(10) Pub. No.: US 2017/0056045 A1
(43) Pub. Date: Mar. 2, 2017

(54) CATHETER EXTRACTION

(71) Applicants: Gabriel Herscu, Livermore, CA (US); Curt Toppel, Manhattan Beach, CA (US); Elmer Tolentino, JR., Livermore, CA (US)

(72) Inventors: Gabriel Herscu, Livermore, CA (US); Curt Toppel, Manhattan Beach, CA (US); Elmer Tolentino, JR., Livermore, CA (US)

(21) Appl. No.: 14/846,989

(22) Filed: Sep. 7, 2015

Related U.S. Application Data
(60) Provisional application No. 62/209,216, filed on Aug. 24, 2015.

(51) Int. Cl. A61B 17/3205; A61M 25/04; A61B 17/3209; A61M 25/01

(52) U.S. Cl. CPC A61B 17/3205; A61M 25/0194; A61M 25/04; A61B 17/3209

(57) ABSTRACT

A catheter extraction tool has a head that can be placed in position to significantly surround a diameter of a catheter. The head is shaped to have a low enough profile above the catheter to allow the head to be slid down the catheter and into a subdermal region in which the catheter is subdermal with respect to a patient. The head of the catheter extraction tool is expanded sufficiently to slide the head over a catheter cuff located in the subdermal region. After the head of the catheter extraction tool slides over the catheter cuff, the head is contracted to engage the catheter so that a user can pull the catheter out of the patient using the catheter extraction tool.

### BACKGROUND

A tunneled central venous catheter is a medical device, placed with the tip of the catheter in the central venous system, allowing for administration of medication, blood draw for laboratory tests, and hemodialysis access. The catheter consists of an intravenous portion and a sub-dermal portion. The sub-dermal portion of the catheter has a catheter cuff or other fixation modality that becomes incorporated into the subcutaneous tissue, forming a “seal” against bacteria or other contaminants. Since a cuff on the catheter is the most common fixation modality, it will henceforth be referred to as “cuff” but may represent any type of fixation modality on the catheter. The common placement for the catheter is on the chest wall, tunneled under the skin and entering the central venous system through the subclavian vein or internal jugular vein. The tip of the catheter is typically positioned in the superior vena cava or right atrium.

In contrast to traditional, non-tunneled catheters, the tunneled catheters provide longer term access due to decreased infection rates and decreased rates of catheter dislodgement. These catheters are considered temporary for acute needs or for dialysis access while permanent dialysis access is being achieved. The catheter is removed when no longer necessary.

Removal of the tunneled catheter requires a small surgical procedure, typically performed in a doctor's office or in a surgery suite. Local anesthetic is administered and surgical dissection along the catheter is performed until the catheter cuff has been disconnected from the surrounding tissue. A “sheath” of scar tissue forms around the tubing in the subcutaneous tissue and this sheath requires division prior to removal of the catheter. The catheter is then pulled from the central venous system by gentle traction.

The surgical nature of catheter removal commonly prompts consultation to a surgical specialist each time the catheter is removed. The procedure itself requires approximately 15 minutes for complete removal including obtaining hemostasis.

### BRIEF DESCRIPTION OF THE DRAWINGS

FIG. 1 is an angled front view of a catheter extraction device in accordance with an implementation.

FIG. 2 is an enlarged front view of a head of the catheter extraction device shown in FIG. 1 clamped around a catheter just distal to a catheter cuff in accordance with an implementation.

FIG. 3 is an angled view of a catheter extraction device in accordance with another implementation.

FIG. 4 is an enlarged view of a sectional view of a head of the catheter extraction device shown in FIG. 3 in accordance with another implementation.

FIG. 5A, FIG. 5B and FIG. 5C show the catheter extraction device shown in FIG. 3 moved over a catheter cuff in accordance with an implementation.

FIG. 6 shows the handle of a catheter extraction device that allows for a rod or wire to be manipulated in order to change a form of a distal head in accordance with another implementation.

FIG. 7A is a side view of the handle of a catheter extraction device shown in FIG. 6 in accordance with an implementation.

FIG. 7B shows a head portion added to the handle of a catheter extraction device shown in FIG. 7A in accordance with an implementation.

FIG. 8 is an enlarged side view of a head of the catheter extraction device shown in FIG. 7B in accordance with an implementation.

FIG. 9A and FIG. 9B present enlarged angled views illustrating the opening of the head of the catheter extraction device shown in FIG. 7B in accordance with an implementation.

FIG. 10 and FIG. 11 illustrate operation of the of the head of the catheter extraction device shown in FIG. 7B in accordance with an implementation.

FIG. 12, FIG. 13, FIG. 14 and FIG. 15 show various alternative implementations of head shapes for a catheter extraction device.

FIG. 16A, FIG. 16B and FIG. 16C show another alternative implementations of a head shape for a catheter extraction device.

### DESCRIPTION OF THE EMBODIMENT

Efficient and expeditious extraction of a catheter is described herein. While, specific embodiments pertain to a tunneled central venous catheter, other types of catheters may be extracted as described herein, including catheters with alternative tunneling or fixation modalities.

Also described is a catheter extraction device that allows removal of a tunneled catheter in less time than existing methods without requiring surgical skill. The catheter extraction device is advanced along the existing catheter until it meets the catheter cuff. The device then passes over the catheter cuff and is used to dissect the catheter cuff away from surrounding tissue. Once past the catheter cuff the device is used to break the sheath of scar tissue surrounding the catheter cuff to separate the catheter from any bodily attachment. Then the catheter extraction device is used to remove the catheter with gentle traction force.

The catheter extraction device and method described herein facilitates removal of catheters by non-surgeons in a hospital or office setting without consultation to a specialist. This provides for less delay in catheter removal as well. It is a less painful procedure for the patient and decreases trauma to the surrounding tissues. In addition, catheter removal procedure time is decreased.

For example, a catheter extraction device is produced in either disposable or reusable forms and is made available to doctor offices and hospitals as an “off the shelf” solution when tunneled catheter removal is necessary.

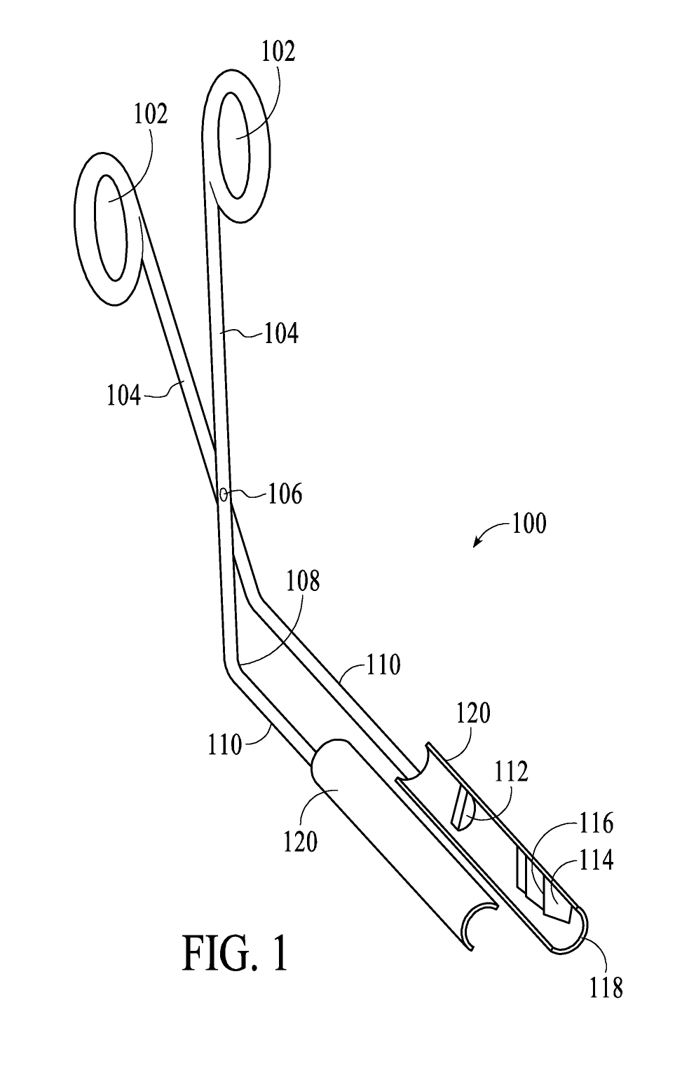

FIG. 1 shows a catheter extraction device 100 that can be opened and closed using the opposing fingers by inserting the opposing fingers into finger holes 102 within a handle of catheter extraction device 100. For example, catheter extraction device 100 also includes pre-joint shafts 104, a joint 106, and post-joint shafts 110. Each post-joint shaft 110 has, for example, a bend 108 at an angle of approximately 15 degrees. Alternatively, for bend 108 any angle from 0 to 179 degrees may be utilized depending upon a specific application.

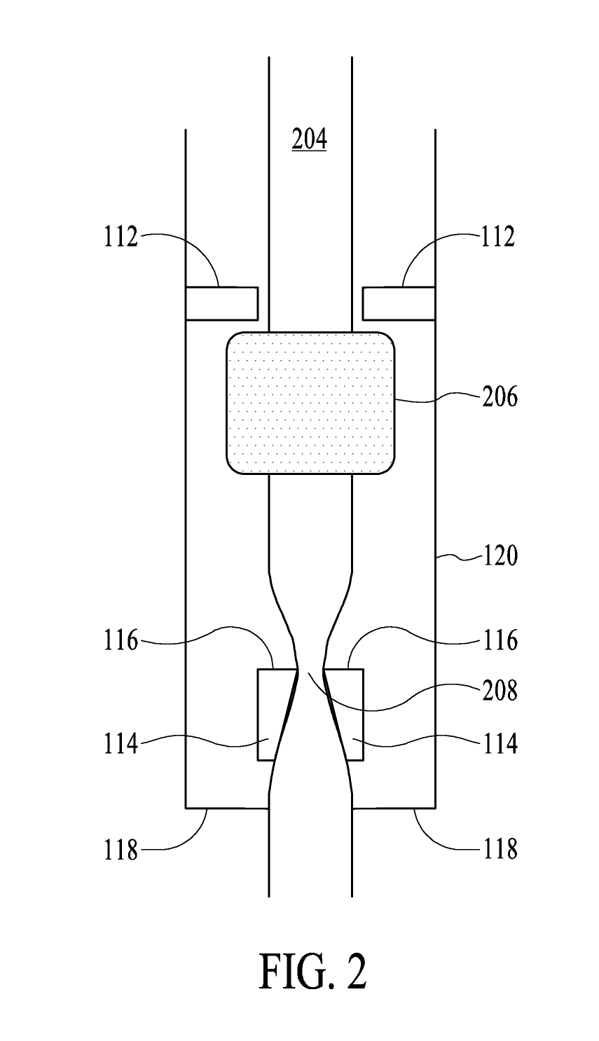

A head of catheter extraction device 100 includes, for example, curved body parts 120 that, when fully closed, fits around a catheter. Each curved body part 120 has, for example, an angled platform 114 on which lies a sharp edge 116. A distal edge 118 of each curved body part 120 is, for example, rounded such that it contours to the catheter and does not cut or damage skin and other tissues. Alternatively, other shapes can be used for distal edge 118. For example, the exact shape of distal edge 118 can be squared, spherical or any other shape that can improve performance. In alternative implementations in addition to the implementation shown in FIGS. 1 and 2, a convex curved head element can be replaced with a locking gear mechanism that can be manipulated through rotation of the handle. Other variations of a blade with catheter cuff grip facility also can be used to facilitate extracting a tunneled central venous catheter.

For example, catheter extraction device 100 is mounted on an existing catheter and advanced through the defect in the skin, where the catheter exits the patient. This is done, for example, by surrounding the catheter with curved body parts 120 of catheter extraction device 100 near the catheter exit site. Curved body parts 120 compress the catheter slightly to allow for a low profile allowing advancement of the head of catheter extraction device 100 through the existing catheter exit site. Then, curved body parts 120 are slid along the catheter (distal edge 118 of the head first) using moderate pressure until distal edge 118 of the head reaches the catheter cuff. The length of post-joint shaft 110 is, for example, long enough to reach past the catheter cuff on any tunneled central venous catheter.

At this point curved body parts 120 are expanded (i.e., the head is opened) in order to dissect the catheter cuff from surrounding tissue and in order to allow for enough room for curved body parts 120 to slide past the catheter cuff. Then, slight pressure is given to the catheter extraction device 100 to push curved body parts 120 past the catheter cuff. Once the angled platform 114 and sharp edge 116 of curved body parts 120 pass the catheter cuff, the catheter extraction device 100 is squeezed lightly and inserted until a backstop 112 on each of curved body parts 120 comes in contact with the catheter cuff. The user squeezes the handle of catheter extraction device 100 so that sharp edges 116 cut any remaining sheath of scar tissue that prevents the catheter from freely sliding out of the patient. The user then firmly squeezes the handle of catheter extraction device 100 and pulls to remove the catheter in its entirety.

FIG. 2 illustrates an enlarged front view of catheter extraction device 100 as it lies in a clasped position past a catheter cuff 206. The backstop 112 on each of curved body parts 120 should preferably rest up against a distal edge of catheter cuff 206, and the curved body should surround a pre-cuff catheter portion 204. As the catheter extraction device 100 is squeezed, angled platform 114 and sharp edge 116, compress a post-cuff catheter portion 208 and preferably also cut through any sheath that may have formed bound to post-cuff catheter portion 208.

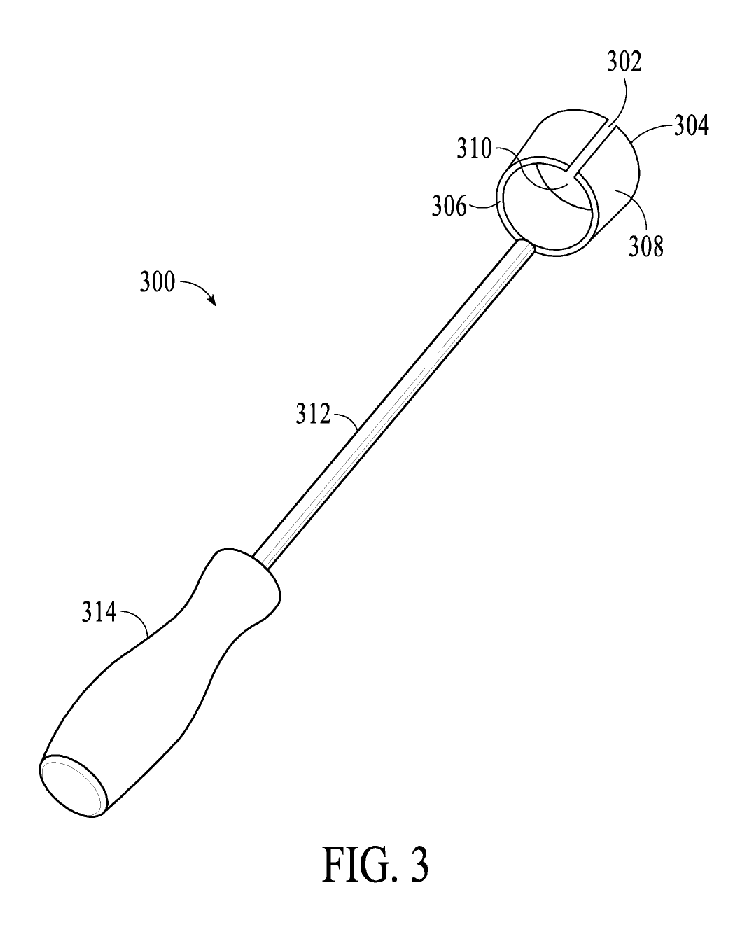

FIG. 3 illustrates another implementation of a catheter extraction device. Catheter extraction device 300 includes a gripping handle 314, a shaft 312, and a cylindrical head composed of a curved body 308 with an opening 302. Curved body 308 surrounds a space 310 into which is placed catheter that is to be extracted. Curved body 308 includes a proximal beveled bladed edge 306 with an approximate circumference of that of the catheter, and a non-sharp distal edge 304 with a slightly larger circumference to accommodate the cuff. For example, curved body 308 is composed of spring steel. Alternatively, other materials can be used such as stainless steel, a polymer plastic, carbon based such as carbon fiber, embedded plastic with metal, autoclave plastic, or some other suitable material.

To extract a catheter from a patient, the catheter is squeezed into opening 302 to load the catheter into space 310. Catheter extraction device 300 is then slid along the catheter through an epidermal opening of the patient until the non-sharp distal edge 304 reaches the catheter cuff.

With pressure, the spring steel of curved body 308 opens at the catheter cuff allowing curved body 308 to pass the catheter cuff and begin dissection of any surrounding tissue from the catheter cuff using non-sharp distal edge 304. Once past the catheter cuff, the spring steel of curved body reverts to its original shape and this force, coupled with a small amount of rotation, allows for beveled bladed proximal edge 306 to engage the subcutaneous sheath, subdermal scar, or any other material, at the catheter cuff and dissect it away from the catheter. Once this tissue has been dissected from any catheter elements, a gentle traction force can be applied to Catheter extraction device 300, removing the catheter from the patient.

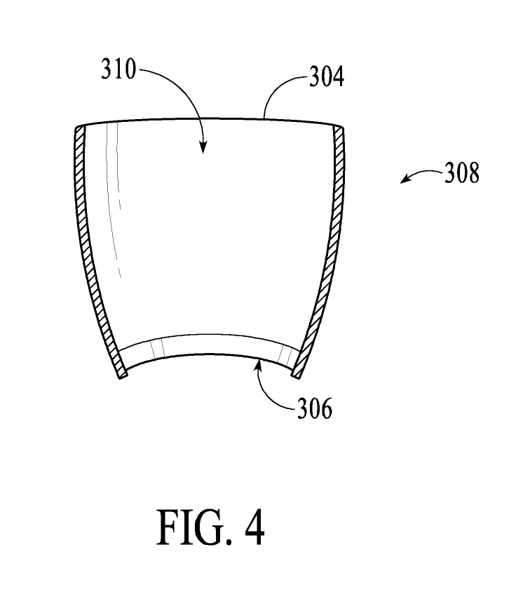

FIG. 4 illustrates an enlarged view of a cross section of the preferred embodiment of the distal circular head in FIG. 3. Curved body 308 surrounds space 310 which has a diameter approximately equal to that of the catheter that is to be removed at the proximal end. Non-sharp distal edge 304 is used to dissect the surrounding tissue as the spring steel of curved body 308 expands around the catheter cuff. The beveled blade proximal edge 306 engages the “sheath” at the catheter cuff and breaks it. In this example, a simple conical body with beveled blade proximal edge is used. However, any conical body, such as a conical inverted j-shape or any other conical shape can be used. These different body shapes can have singular or multiple blades affixed to their edges. In some instances, the body can further have internal diameter variance. This variance allows for optimizing the capturing of surrounding tissue for dissection.

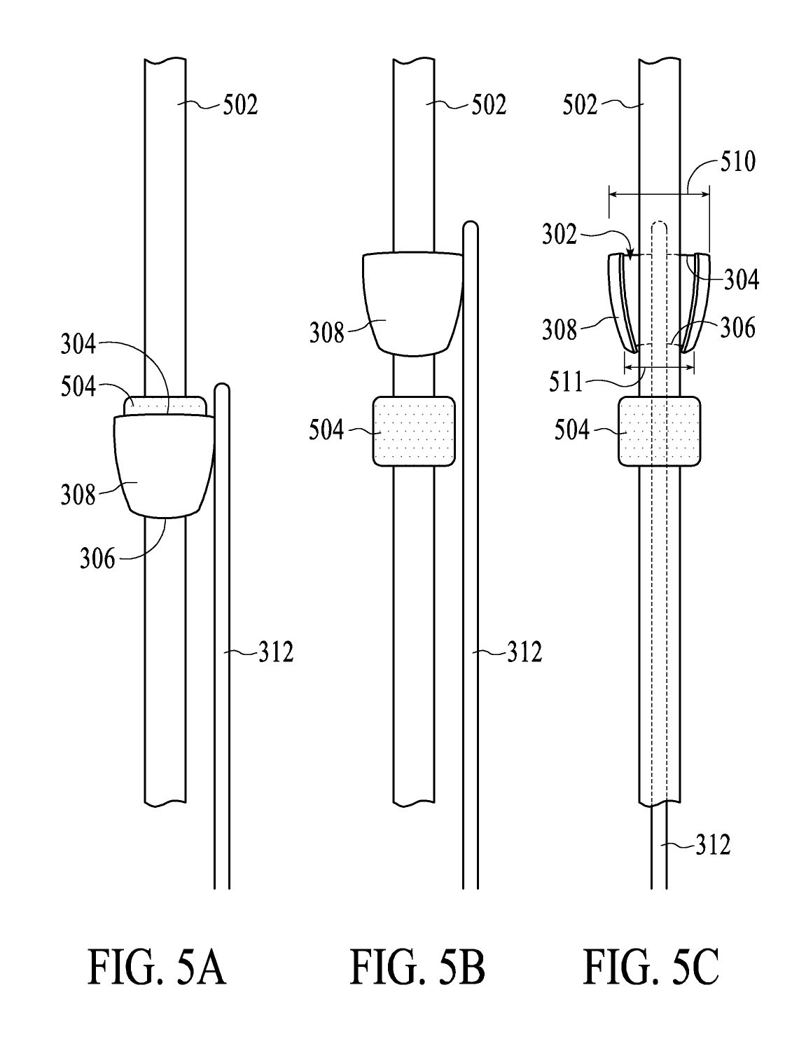

FIG. 5A and FIG. 5B illustrate how curved body 308 attached to the rod 312 moves over and past catheter cuff 504 on catheter 502. As the larger and distal edge 304 of curved body 308 hits catheter cuff 504, space 302 between within two parts of curved body 308 widens allowing for proximal edge 306 to pass over the catheter cuff 504. FIG. 5C illustrates how when rod 312 is used to withdraw catheter extraction device 300, diameter 511 of beveled blade proximal edge 306 is reduced relative to diameter 510 of beveled blade proximal edge 306 allowing curved body 308 to engage catheter cuff 504 and pull catheter 502 out from the patient.

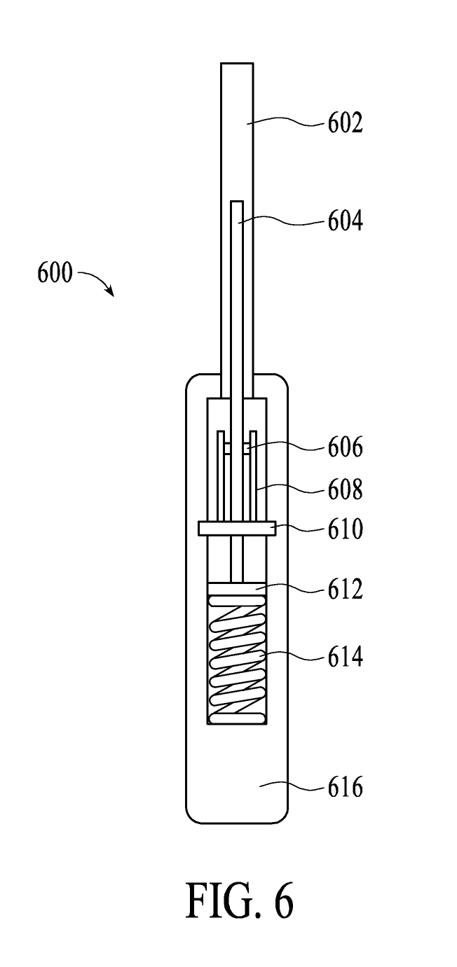

FIG. 6 illustrates another implementation of a catheter extraction device. Catheter extraction device handle portion 600 includes a handle 616 and a shaft 602 for a rod 604. Rod 604 has a base 612 and a small bar 606, which is attached to a large bar 610 using a flat connector 608. A spring 614 is located within handle 616.

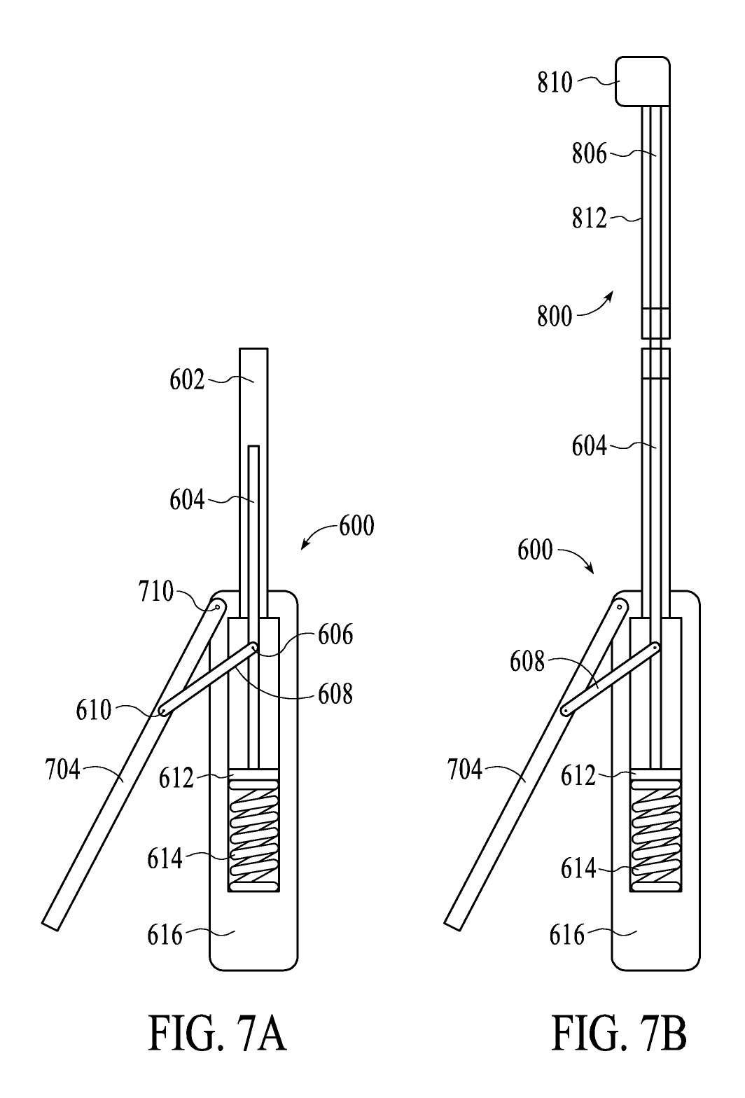

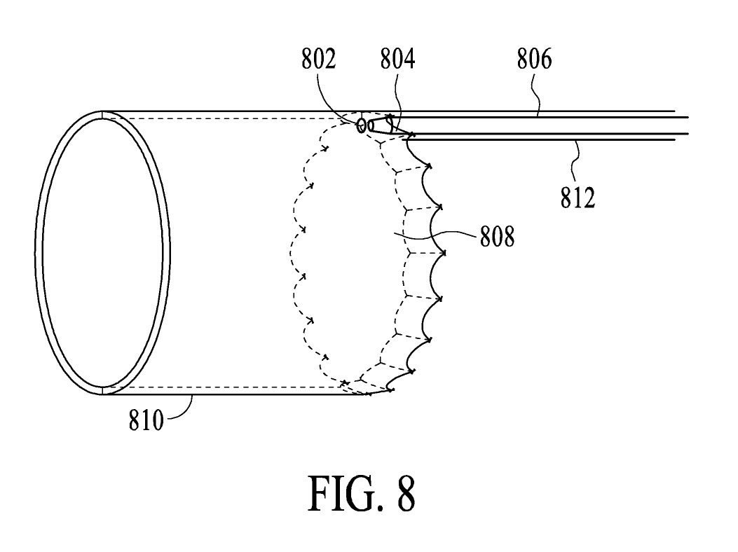

As shown in FIG. 7A, a lever 704 has been attached to handle 616 at a joint 710. Lever 704 is connected to flat connector 608 at large bar 610. The location of small bar 606 is also shown in FIG. 7A. Note that in this example 606 and 610 are bars but joints can also be used. Force can be applied to lever 704 in order to rotate lever 704 at joint 710 and bring lever 704 closer to handle 616. This force and movement of lever 704 preferably manipulates large bar 610 which will then in turn manipulate small bar 606 as small bar 606 is connected with flat connector 608. This action preferably moves rod 604 down shaft 602 to open an attached head on a distal end. Spring 614 attached to the base 612 is preferably extended during this action as well. Once force is removed from lever 704 attached to handle 616, spring 614 preferably recoils causing the rod 604 to return to its original position by sliding back down shaft 602. In this example, a lever with spring system is used to actuate the system. However, in some instances, a ratcheted mechanism can perform the same task. A ratchet system differs slightly in that actuation moves the rod an incremental amount forward, opening the head. Successive ratchet movements further the rod down the shaft until such time the user has opened the head to its widest diameter. When set in reverse, ratchet movements move the rod back down the shaft and closes the head.

FIG. 7B illustrates how catheter extraction device head attachment 800 attachment can be fitted onto catheter extraction device handle portion 600. Rod 806 in catheter extraction device heat attachment 800 can be manipulated by rod 604 of catheter extraction device handle portion 600 and causes a head 810 attached to a shaft 812 to open allowing for head 810 to pass over a catheter cuff on a catheter.

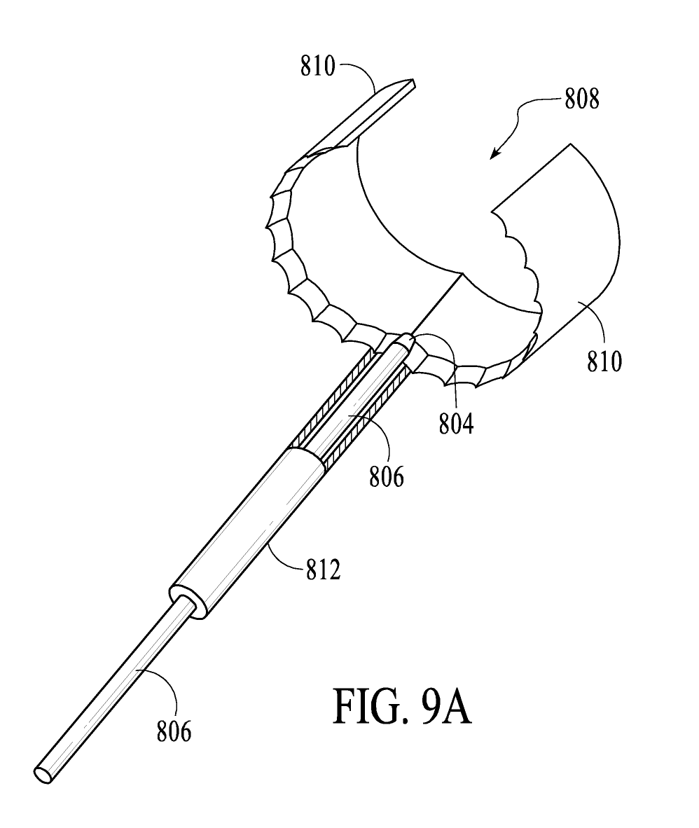

FIG. 8 illustrates how a tip 804 of rod 806 enters into a slot 802 on head 810, which then widens a space 808 allowing for head 810 to pass over the catheter cuff.

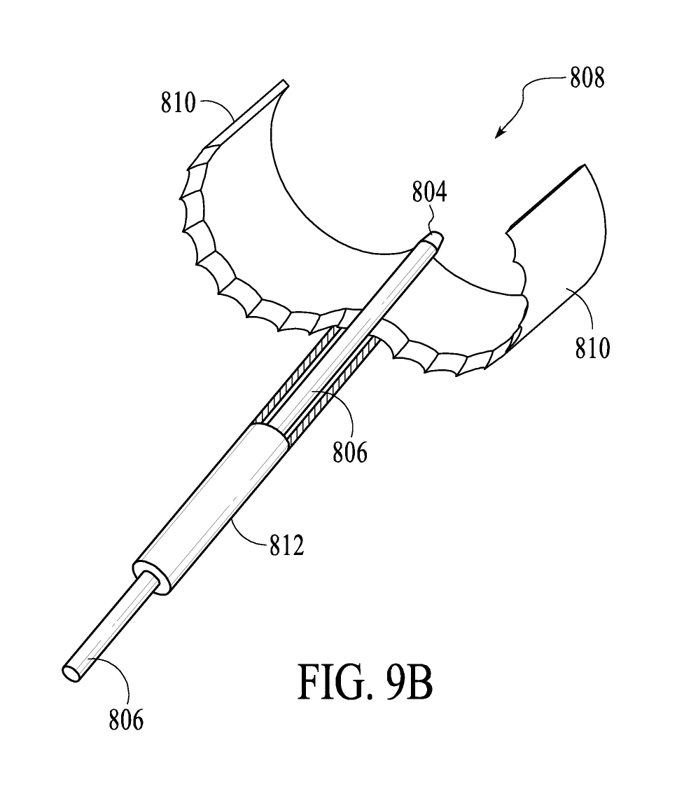

FIG. 9A and FIG. 9B illustrate an enlarged cut-out view that illustrates the dynamics of the opening of head 810. As rod 806 is pushed down the shaft 812, tip 804 preferably pushes at the bases of the left piece of head 810 and the right piece of head 810 surrounding space 808. Upon further force, pieces of head 810 preferably widen in angle and increasing space 808 which will allow for head 810 to dissect any tissue from the catheter cuff and pass over it.

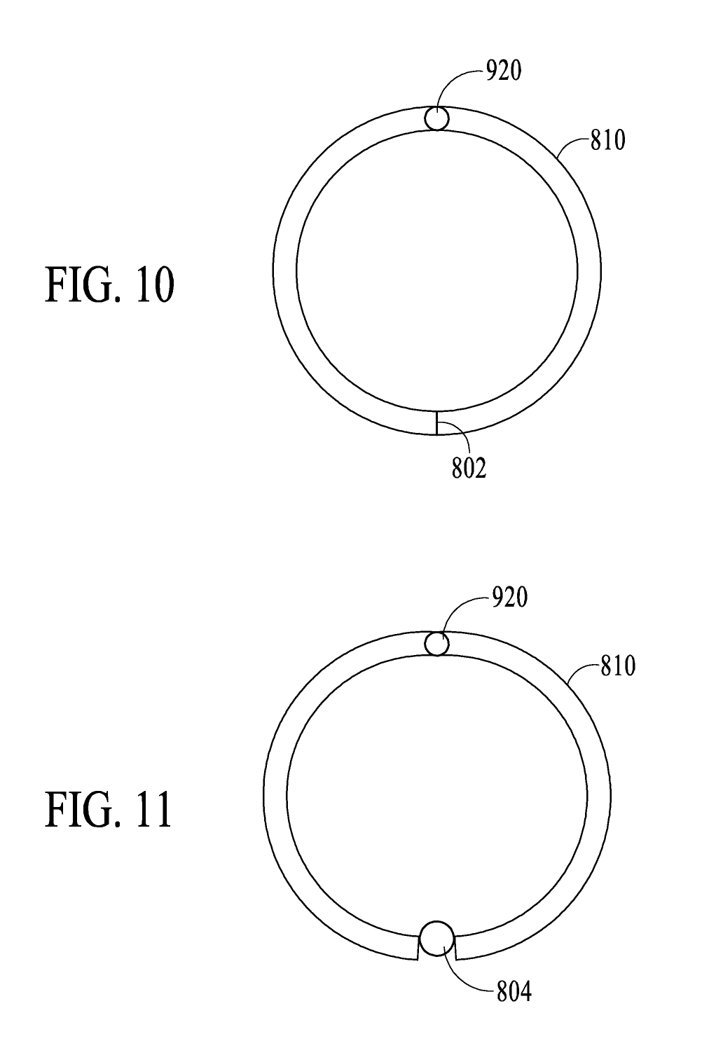

FIG. 10 and FIG. 11 further illustrate operation of head 810. Head 810 employs a catheter gripping facility with subdermal scar cutting capability. Head 810 is composed of two cylindania connected by a pivot pin with an embedded torsion spring 920. In its normal state, the torsion spring applies torque that continuously forces both cylindania at a torque axis point to form a closed cylinder as illustrated by FIG. 10.

To open head 810, a user actuates lever 704 to move a rounded end tip 804 of rod 806 so that rounded end tip 804 meets a notched opening 802 of head 810. As shown by FIG. 11, as rounded end tip 804 of rod 806 continues to be moved towards and through head 810, the cylindania of head 810 open to allow room for rod 806 to pass through.

For example, a user can continue to actuate lever 704 until head 810 opens a desired amount, for example, approximately 3 millimeters. In this example, 3 millimeters has been used, but any distance that sufficiently allows head 810 to pass the catheter cuff can be used. Once head 810 passes the catheter cuff, the user will release grip on lever 704 to reverse, allowing rod 806 to be withdrawn from head 810 so that embedded torsion spring 920 will cause the cylindania of head 810 to revert back to the closed position shown in FIG. 10. At this point, subdermal scar adhering to any catheter elements may prevent removal of the catheter from the patient.

For example, head 810 is shaped similar to a head 1010 shown in FIG. 13. To remove subdermal scar adhering to any catheter elements, the proximal edge of head 810 contains within an inner diameter a serrated edge (similar to a serrated edge 1012 shown in FIG. 13). While a user manipulates handle 616 using any combination of pulling, pushing and or rotating motions, any adherent scar will be cut away from the catheter. In addition, the distal edge of head 810 can use an angled or beveled serrated edge (similar to a serrated edge 1011 shown in FIG. 13) for further tissue dissection while traversing towards catheter cuff 504 using the same motions. Once the catheter is freed from the scar tissues, the user will be able to extract the catheter from the patient.

In the above example implementations, a symmetrical cylindrical head is depicted but any shape that improves performance is also disclosed. In all cases, exterior surfaces are smooth and rounded.

Also, the rod system shown in FIGS. 6 through 11 may be interchanged with a wire system that allows for the same purpose of opening the head of a catheter extraction device. The spring mechanism shown in FIGS. 6 through 11 can also be interchanged with any other mechanism, such as a ratcheted system, used to bring the catheter extraction device 100 back to its original position after force has been removed from a lever.

Also, catheter line diameters may vary based on application. Dimensions of the heads of the catheter extractions devices described above can be varied to fit any tunneled central venous catheter implementations.

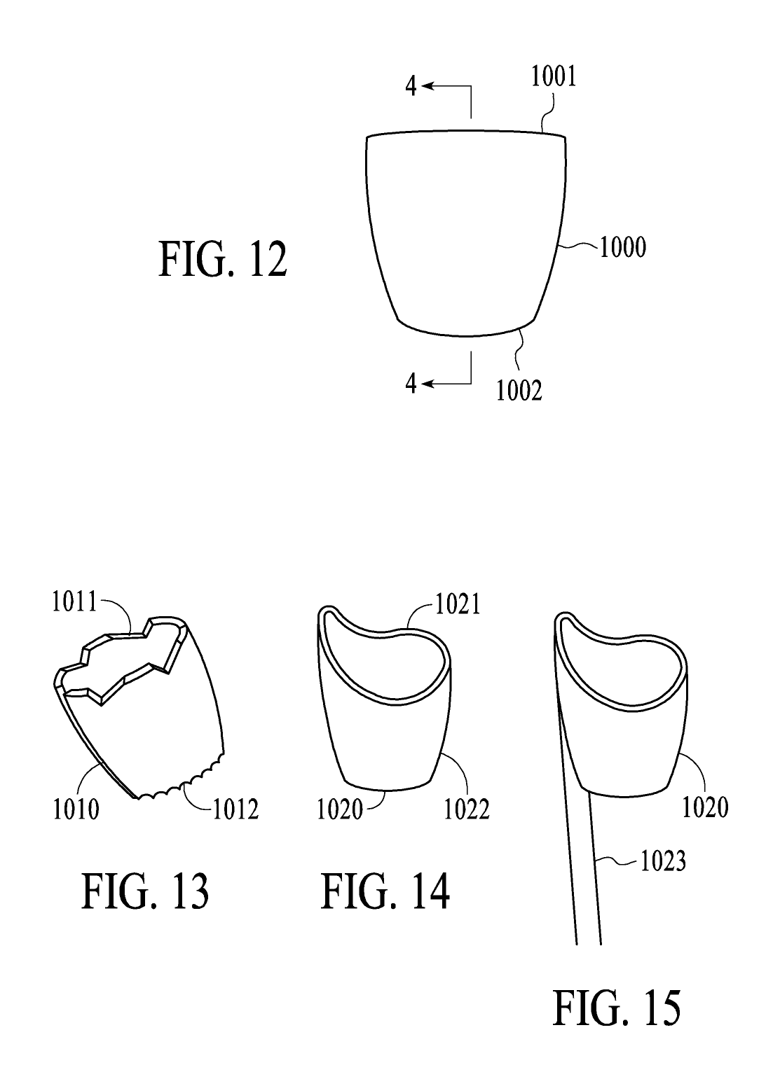

FIG. 12, FIG. 13, FIG. 14, and FIG. 15 show various alternative implementations of head shapes for a catheter extraction device. FIG. 12 illustrates that a diameter at an edge 1001 of a head 1000 is larger than a diameter at an edge 1002. After edge 1001 is pushed over a catheter cuff, head 1000 expands so that edge 1002 also travels over the catheter cuff before head 1000 contracts allowing edge 1002 to engage with the catheter cuff.

FIG. 13 shows edge 1011 of head 1010 being a serrated cutting edge to allow for the head to separate the catheter and the catheter cuff from any bodily attachment. Edge 1012 can also be serrated. Serrated or sharpened edges may be placed on any surfaces of the heads shown in FIGS. 12, 13, 14 to produce cutting edges that provide cutting ability to dissect the cuff from the tissue and/or cut the scar sheath to remove the catheter.

FIG. 14 shows an edge 1021 of a head 1010 being a cutting edge to allow for the head to separate the catheter and the catheter cuff from any bodily attachment. Edge 1022 can also be serrated. FIG. 15 shows a shaft 1023 connected to head 1020.

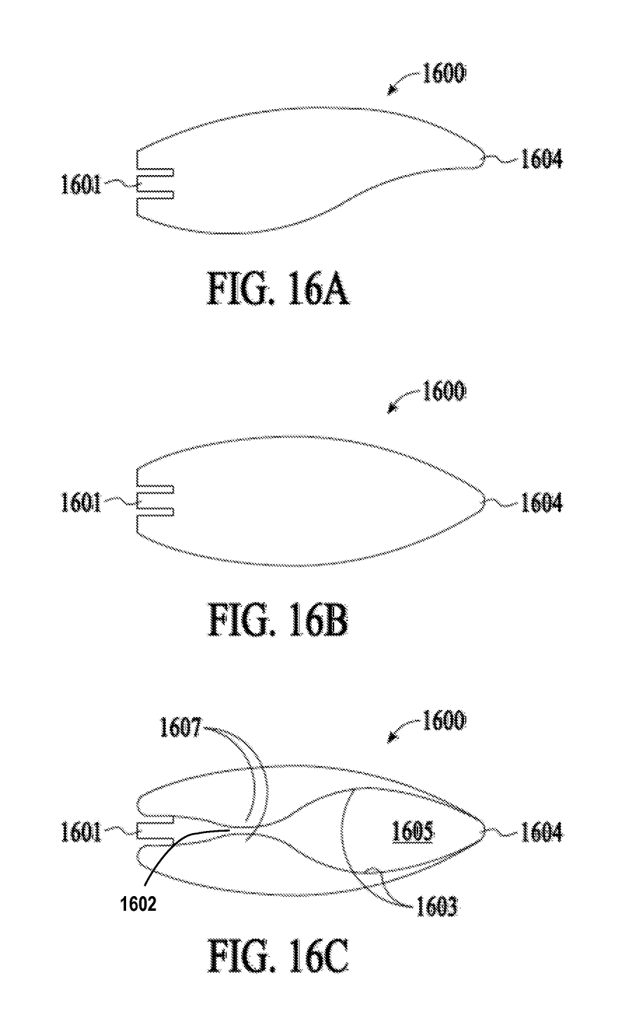

FIG. 16A, FIG. 16B and FIG. 16C show various views of another preferred embodiment of a head 1600. Head 1600 consists of a preferably pointed tip 1604, cutting edges 1603 that line distal ends of wings 1607, which surround a space 1605. Preferably included is also a catheter insertion point 1602 and flexible end pieces 1601. The catheter is preferably inserted at the catheter insertion point 1602 until surrounded by wings 1607, and able to move through space 1605. Using a shaft, head 1600 is preferably pushed along the catheter and past the dermal entry until it reaches the cuff on the catheter. Wings 1607 hold the catheter securely within during the motion.

Once head 1600 is subdermal, it is preferably guided by the shaft until it hits the catheter cuff. At this point, pointed tip 1604 is used to penetrate the tissue surrounding the catheter cuff and pass it. Most likely, the ratcheted system would be used to move head 1600 in a controlled manner past the cuff but any system could be used. For example, an axial rotation and twisting motion can be used while continually exerting forward force. Cutting edges 1603 are preferably used to cut most of or all of the tissue surrounding the catheter cuff. Wings 1607 can be rigid or slightly flexible and open slightly as they pass over the cuff. The flexible end pieces 1601 preferably widen allowing the entirety of head 1600 to pass over the catheter cuff. Once past the catheter cuff, flexible end pieces 1601 narrow again and put pressure inwards onto the scar sheath surrounding the catheter past the catheter cuff. The user can then pull back on the shaft and device which allows for the flexible end pieces 1601 to grip and cut some or all of the scar sheath allowing for the catheter to be removed using traction. During the pulling force, head 1600 and/or the flexible end pieces 1601 can also preferably evert allowing for a more perpendicular cutting force to be applied to the scar sheath surrounding the catheter.

A motorized version of the catheter extraction tool can be used where the head is connected to a rotating or oscillating motor element. This can allow for more effective dissection of tissues during cuff dissection or scar sheath division.

The foregoing discussion discloses and describes merely exemplary methods and embodiments. As will be understood by those familiar with the art, the disclosed subject matter may be embodied in other specific forms without departing from the spirit or characteristics thereof. Accordingly, the present disclosure is intended to be illustrative, but not limiting, of the scope of the invention, which is set forth in the following claims.

What is claimed is:

1. A catheter extraction tool comprising:
a head that can be placed in position to significantly surround a diameter of a catheter, the head being shaped to have a low enough profile above the catheter to allow the head to be slid down the catheter and into a subdermal region in which the catheter is subdermal with respect to a patient; and,
a user interface portion that allows a user to expand the head of the catheter extraction tool sufficiently to slide the head over a catheter cuff located in the subdermal region, the user interface portion also allowing the user to contract the head of the catheter tool so that after the head of the catheter extraction tool slides over the catheter cuff, the head can be contracted to engage the catheter so that the user can pull the catheter out of the patient using the catheter extraction tool.

2. A catheter extraction tool as in claim 1, wherein the user interface portion includes:
a first shaft connected to a first half of the head;
a second shaft connected to a second half of the head;
a joint between the first shaft and the second shaft;
a first pre-joint shaft connected to the first shaft;
a second pre-joint shaft connected to the second shaft;
a first finger hole attached to the first pre-joint shaft; and,
a second finger hole attached to the second pre-joint shaft;
wherein using fingers inserted in the first finger hole and the second finger hole, the user expands and contracts the head of the catheter extraction tool by adjusting the distance between the first finger hole and the second finger hole.

3. A catheter extraction tool as in claim 2:
wherein the first half of the head includes an angled platform on which lies a sharp edge that is used to engage the catheter when the head is contracted; and,
wherein the second half of the head includes an angled platform on which lies a sharp edge that is used to engage the catheter when the head is contracted.

4. A catheter extraction tool as in claim 2:
wherein the first half of the head includes a backstop that rests against the catheter cuff when the catheter is removed; and,
wherein the second half of the head includes a backstop that rests against the catheter cuff when the catheter is removed.

5. A catheter extraction tool as in claim 1, wherein the user interface portion includes:
a gripping handle; and,
a shaft connected between the gripping handle and the head;
wherein the head is composed of expandable material and shaped with a wider diameter at a top where the head first meets the catheter cuff, so that as the user pushes the head down the catheter, when the head encounters the catheter cuff, pressure from the user on the gripping handle forces the expandable material to expand allowing the head to expand sufficiently to travel over the catheter cuff, the expandable material contracting upon the user finishing pushing the head over the catheter cuff.

6. A catheter extraction tool as in claim 5, wherein the head additionally includes cutting elements that facilitate dissection of subdermal disuse by rotation of the catheter dissection tool

7. A catheter extraction tool as in claim 1, wherein the user interface portion includes:
a gripping handle; and,
a shaft connected between the gripping handle and the head;
a rod that travels within the shaft;
a lever that the user uses to control travel of the rod within the shaft;
wherein when the rod travels in a first direction within the shaft, this forces the head to expand, and when the rod travels in a second direction within the shaft, this allows the head to contract.

8. A catheter extraction tool as in claim 7, additionally comprising:
a spring within the gripping handle, the spring being attached to the rod, the spring exerting pressure to bring the rod to a default position within the shaft when the lever is used to push the rod away from the default position within the shaft.

9. A catheter extraction tool as in claim 7, wherein the head additionally includes cutting elements that facilitate dissection of subdermal disuse by rotation of the catheter dissection tool.

10. A catheter extraction tool as in claim 7, wherein the lever controls travel of the rod within the shaft using a ratcheting mechanism that uses ratchet movements to control travel of the rod within the shaft.

11. A catheter extraction tool as in claim 1, wherein the head includes a cutting edge that can be used to separate the catheter and the catheter cuff from any bodily attachment.

12. A catheter extraction tool as in claim 1, wherein the head includes multiple cutting edges that can be used to separate the catheter and the catheter cuff from any bodily attachment.

13. A catheter extraction tool as in claim 1 additionally comprising:
a motor element that allows rotates or oscillates the head for more effective dissection of tissues during cuff dissection or scar sheath division.

14. A method to extract a catheter from a patient, comprising:
placing a head of a catheter extraction tool in position to significantly surround a diameter of a catheter;
sliding the head down the catheter and into a subdermal region in which the catheter is subdermal with respect to the patient;
expanding the head of the catheter extraction tool sufficiently to slide the head over a catheter cuff located in the subdermal region;
contracting the head of the catheter tool, after the head of the catheter extraction tool slides over the catheter cuff, to engage the catheter; and,
pulling the catheter out of the patient using the catheter extraction tool.

15. A method as in claim 14, wherein contracting the head of the catheter tool to engage the catheter includes:
contracting a first half of the head to a second half of the head so that a sharp edge of the first head and a sharp half of the second head engages the catheter.

16. A method as in claim 14, wherein expanding the head of the catheter extraction tool includes:
pushing the head down the catheter so that when the head encounters the catheter cuff, pressure from on a gripping handle forces expandable material of the head to expand sufficiently for the head to travel over the catheter cuff.

17. A method as in claim 14, wherein expanding the head of the catheter extraction tool includes:
using a lever to control travel of a rod within a shaft connected between a gripping handle of the catheter extraction tool and the head, wherein when the rod travels in a first direction within the shaft, this forces the head to expand, and when the rod travels in a second direction within the shaft, this allows the head to contract.

18. A method as in claim 14 additionally comprising:
using a spring within the gripping handle to exert pressure to bring the rod to a default position within the shaft when the lever is used to push the rod away from the default position within the shaft.

19. A method as in claim 14 additionally comprising:
using a cutting edge on the head to separate the catheter and the catheter cuff from any bodily attachment.

20. A method as in claim 14 additionally comprising:
using multiple cutting edges on the head to separate the catheter and the catheter cuff from any bodily attachment.
